(19) United States
(12) Patent Application Publication
(10) Pub. No.: US 2025/0271417 A1
(43) Pub. Date: Aug. 28, 2025

(54) METHOD FOR DETERMINING WHETHER A BOVINE HAS A RESPIRATORY DISEASE

(71) Applicant: Agscent Pty Ltd, Carwoola NSW (AU)

(72) Inventors: Bronwyn Darlington, Carwoola NSW (AU); Jessica Darlington-Brown, Carwoola NSW (AU)

(21) Appl. No.: 18/858,194

(22) PCT Filed: May 17, 2023
(86) PCT No.: PCT/AU2023/050311

§ 371 Date: Oct. 18, 2024

(30) Foreign Application Priority Data
Apr. 20, 2022 (AU) 2022901042

(51) Int. Cl. G01N 33/497; A61B 5/08; G01N 30/02; G01N 30/72; G01N 30/86; G01N 30/88

(52) U.S. Cl. CPC G01N 33/4975; A61B 5/082; G01N 30/7206; G01N 30/8679; G01N 2030/025; G01N 2030/884; G01N 2800/26

(57) ABSTRACT

Disclosed herein is a method for determining whether a bovine has a respiratory disease such as bovine respiratory disease (BRD). The method comprises detecting an amount of one or more biomarkers in a breath sample from the bovine, the detected amount of the or each biomarker defining a detected biomarker profile, and comparing the detected biomarker profile to a predetermined biomarker profile, whereby a correlation between the detected and predetermined biomarker profiles is indicative of the bovine having the respiratory disease.

### TECHNICAL FIELD

The present invention relates to methods and sensors for determining whether a bovine has a respiratory disease.

### BACKGROUND ART

Bovine animals, such as domestic cattle, can suffer from a number of respiratory diseases. Bovine respiratory disease (BRD), for example, is one of the most common and costly diseases that affect cattle. In Australia, for example, BRD is the most common disease affecting feedlot cattle, with annual losses estimated at approximately AU$60 million. Morbidity, mortality, loss of feed resources, medication purchases, increased time on feed and associated labour costs all contribute to the economic loss caused by BRD. In addition to costs associated with performance loss and animal death, the cattle industry spends millions of dollars each year attempting to prevent and treat BRD. Early diagnosis and treatment of BRD is thought to improve prognosis and outcome, but late diagnosis and therapy is more likely to result in treatment failure.

BRD is a complex progressive disease in which a number of viral and bacterial pathogens interact with environmental factors, all of which contribute to the disease's susceptibility. Because there are multiple infectious aetiologies, diagnosing BRD can be challenging.

Currently, BRD is diagnosed using a variety of approaches, with ultrasound being one of the most commonly used methods. Thoracic ultrasonography is a non-invasive ancillary method for evaluating lung lesions, which can provide additional information to more traditional lung function testing. However, ultrasound examinations are expensive and require a high level of training to perform. The use of PCR for BRD diagnosis is becoming increasingly prevalent, as it allows for the identification of different bacteria and viruses, giving clinicians a broader perspective of the pathogens involved and, as a result, more options for treatment, management, and prevention. However, the availability of laboratories capable of performing PCR analysis is a major limitation.

It would be of significant advantage to the cattle industry if alternative methods for diagnosing respiratory diseases such as BRD were available. Methods that provide for a timely diagnosis and hence enable a quick adoption of potentially more efficacious therapies (e.g. appropriate antimicrobial utilization, based on a positive diagnosis) in order to improve animal wellbeing would be of significant commercial benefit.

### SUMMARY OF INVENTION

The inventors of the invention the subject of the present application have discovered that the breath of bovines infected with bovine respiratory disease contains biomarkers which are characteristic of the disease. As will be described in further detail below, the inventors have been able to demonstrate that a non-invasive breath sampling method (which only takes a few seconds) can be used to determine whether a bovine has bovine respiratory disease. The inventors believe that the results of their preliminary trials, and the suspected reasons for the presence of these biomarkers in the bovine's breath, supports a reasonable prediction that the disclosed methods have applicability for the detection of respiratory diseases in addition to bovine respiratory disease.

In a first aspect, the present invention provides a method for determining whether a bovine has a respiratory disease (e.g. bovine respiratory disease, bovine viral diarrhea or bovine pestivirus). The method comprises detecting an amount of one or more biomarkers in a breath sample from the bovine, the detected amount of the or each biomarker defining a detected biomarker profile, and comparing the detected biomarker profile with ambient air and to a predetermined biomarker profile, whereby a correlation between the detected and predetermined biomarker profiles is indicative of the bovine having the respiratory disease.

In a second aspect, the present invention provides a method for screening a population of bovines for the presence of a respiratory disease. The method comprises detecting an amount of one or more biomarkers in an air sample taken in close proximity to the population of bovines, the detected amount of the or each biomarker defining a detected biomarker profile, and comparing the detected biomarker profile with ambient air and to a predetermined biomarker profile, whereby a correlation between the detected and predetermined biomarker profiles is indicative of one or more bovines in the population having the respiratory disease.

The inventors' proof of concept research and trials have identified biomarkers in the form of volatile organic compounds which either increase or decrease in the breath of cattle having BRD, compared to that in the breath of healthy cattle. Given the progressive nature of BRD, as well as its complex aetiology, it was surprising to the inventors that a consistent biomarker profile was observed in infected animals. The inventors expect that further research (currently underway) may also lead to a correlation between the stage and severity of disease and the relative amounts of the one or more biomarkers in the breath samples. Thus, the present invention has the potential to provide a non-invasive and cost-effective screening option for all stages of BRD using breath analysis. Being able to screen all animals quickly and potentially in an automated fashion would provide a significant positive benefit in the management of BRD. For example, by breath screening for BRD markers at induction of all cattle to a new farm, separation of potentially infected animals could occur prior to integration with other non-infected animals.

Furthermore, as will be described in further detail below, some of the biomarkers identified by the inventors are understood to relate to a bovine's immune response to vaccination. The inventors speculate, and hope to establish with further trials, that such biomarkers may be indicative of an infection with respiratory diseases other than BRD. For example, the inventors' preliminary data supports the detection of bovine pestivirus from a bovine's breath sample.

The identification of a biomarker or biomarkers in an animal's breath, which can be collected using a simple device such as that disclosed in the applicant's international (PCT) patent application no. PCT/AU2020/050318 (WO 2020/198790), the contents of which are incorporated herein, and which is/are indicative of the animal having a respiratory disease such as BRD is a discovery of significant economic benefit. Breath samples may, for example, be taken from animals at a convenient time and, given their inherently non-invasive nature, be carried out on-farm without the need for specialist veterinary expertise.

In some embodiments, the predetermined biomarker profile may, for example, comprise an accumulation of detected amounts of the one or more biomarkers in breath samples from bovines having been diagnosed with the respiratory disease (i.e. using conventional methods). Thus, and as will be described in further detail below, data regarding the presence and amount of the relevant biomarker(s) in the animal's breath can be compared to a biomarker profile of bovines known to be infected. If a correlation is found, then the breath sample is indicative of the bovine being infected with the respiratory disease, whereupon appropriate action (e.g. quarantining from healthy animals in the herd) can be taken.

In some embodiments, detecting an amount of the one or more biomarkers may comprise chromatographically resolving the breath sample. The breath sample may, for example, be chromatographically resolved by gas chromatography. The breath sample may, for example, be chromatographically resolved by gas chromatography in combination with other techniques such as mass spectroscopy (i.e. GC-MS), where mass spectra for a plurality of points of the chromatographically resolved breath sample produces a mass spectral dataset which defines the detected biomarker profile. In such embodiments, the mass spectral dataset produced may be analysed to determine if the bovine has the respiratory disease.

In some embodiments, the mass spectral dataset may be analysed using a multivariate analysis such as partial least squared discriminant analysis (PLS-DA, described in further detail below).

In alternative embodiments, detecting an amount of the one or more biomarkers may comprise contacting the breath sample with a plurality of sensors that are configured to sense the one or more biomarkers, whereby a cumulative response of the sensors is indicative of the bovine having the respiratory disease. As will be described below, “Electric Nose” sensors that are configurable to quickly perform on-site analysis of gasses are commercially available and the inventors expect that such sensors will have utility in the present invention. As would be appreciated, a portable breath screening device which detects respiratory diseases such as BRD in a non-invasive, accurate and economically viable fashion would likely be of great interest to veterinarians, dairy farmers, primary producers and graziers.

In some embodiments, the one or more biomarkers are selected from one or more of the following: 2-nitropropane, furan, acetic acid, ethoxy-1-methylethyl ester, propanoic acid ester, ethylamine and cathinone.

In some of such embodiments, the amounts of one or more of 2-nitropropane, furan, acetic acid, ethoxy-1-methylethyl ester and propanoic acid ester is elevated in the breath of bovine having the respiratory disease. In some of such embodiments, the amount of one or more of ethylamine and cathinone is reduced in the breath of bovine having the respiratory disease.

In some embodiments, at least one of the one or more biomarkers may be a metabolite of a metabolic process affected by the disease state of the animal. In such embodiments, an amount of the at least one biomarker in the breath sample may increase because it is (they are) a metabolite of a metabolic process that is upregulated in sick animals. In such embodiments, an amount of the at least one biomarker in the breath sample may decrease because it is (they are) a metabolite of a metabolic process that is downregulated in sick animals.

In a third aspect, the present invention provides sensor for determining whether a bovine has a respiratory disease. The sensor comprises a detector for detecting an amount of one or more biomarkers in a breath sample from the bovine, and an analyser for analysing the detected one or more biomarkers and comparing with a predetermined biomarker profile, whereby a correlation between the detected one or more biomarkers and the predetermined biomarker profile is indicative of the bovine having the respiratory disease.

In some embodiments, the detector may comprise a plurality of sensors that are configured to detect the one or more biomarkers, whereby a cumulative response of the plurality of sensors is indicative of the bovine having the respiratory disease.

In some embodiments, the sensor of the third aspect of the present invention may be used in the method of the first or second aspect of the present invention.

Other aspects, features and advantages of the present invention will be described below.

### BRIEF DESCRIPTION OF THE DRAWINGS

Embodiments of the present invention will be described in further detail below with reference to the following drawings, in which:

FIG. 1 is a score plot of ambient air and bovines' breath samples, obtained using an OPLS model;

FIG. 2 is heat map of significant biomarkers found in bovines' breath samples depicting up and down regulation of the biomarkers;

FIG. 3 is score plot of case (i.e. diseased) versus control (i.e. non-diseased) bovines' breath samples, obtained using an OPLS model; and

FIG. 4 is a heat map of biomarkers of significance for BRD.

### DETAILED DESCRIPTION OF THE INVENTION

The overarching purpose of the present invention is to determine whether a bovine has a respiratory disease, such as BRD, using methods that either do not require invasive procedures, veterinarians or specialised equipment, or which are quicker and/or more reliable than conventional methods (e.g. those described above). The inventors hope that the results of their ongoing trials and experiments may even enable for a determination of the stage and severity of disease, perhaps even using handheld devices on-farm.

In a first aspect therefore, the present invention provides a method for determining whether a bovine has a respiratory disease. The method comprises detecting an amount of one or more biomarkers in a breath sample from the bovine, the detected amount of the or each biomarker defining a detected biomarker profile, and comparing the detected biomarker profile with ambient air and to a predetermined biomarker profile, whereby a correlation between the biomarker profiles is indicative of the bovine having the respiratory disease.

In a second aspect, the present invention provides a method for screening a population of bovines for the presence of a respiratory disease. The method comprises detecting an amount of one or more biomarkers in an air sample taken in close proximity to the population of bovines, the detected amount of the or each biomarker defining a detected biomarker profile, and comparing the detected biomarker profile with ambient air and to a predetermined biomarker profile, whereby a correlation between the biomarker profiles is indicative of one or more bovines in the population having the respiratory disease.

As noted above and as will be described in further detail below, the inventors have discovered that the breath of bovines contains biomarkers in the form of volatile organic compounds (VOCs) which are characteristic of the bovine having BRD. Breath VOCs are derived from blood flowing through the lungs and therefore can reveal insights into metabolic and pathologic processes occurring elsewhere in the body. The amounts of these biomarkers change when the bovine becomes infected, with these changes possibly being correlatable with progression of the disease. The inventors postulate that the changes in the amounts of the biomarkers which they have discovered to be related to the bovine's disease state may be due to changes such as metabolic changes, physical changes or hormonal changes which occur in the animal's bodies when they become sick. Alternatively, or in addition, the presence of certain virus and bacteria in the bovine may result in the upregulation or downregulation of metabolic pathways that produce the relevant biomarkers.

In the context of BRD, on which the majority of the inventors' attention has been focused thus far, differentiating between infection and disease (stage and severity of pathology) are required to ensure appropriate antimicrobial use in the treatment of BRD. As will be described in further detail below, the inventors' work thus far has involved the collection and analysis of breath samples from healthy and infected animals in feedlots in regional New South Wales, Australia. The initial research was limited by sample size, but the outcomes were very promising, and a number of VOCs have been identified that are higher in infected cattle and there are two VOCs which are lower in infected cattle. Given the complexity of BRD, particularly as it progresses, it was surprising and unexpected to the inventors that a consistency in biomarker profile was observable across all of the bovines' breath samples.

The breath sample may be collected in any suitable manner, using any suitable apparatus. Apparatus similar to those disclosed in PCT/AU2020/050318 may, for example, be used to collect such samples.

The breath sample may be collected from the animal using any suitable technique. In order to reduce the risk of contamination (e.g. by food or saliva), the breath sample may, for example, be collected from the animal's nose.

In practice, breath samples may be taken from the bovine at any suitable interval or at the convenience of the primary producer. Given the significantly higher costs associated with maintaining sick animals, however, it is expected that breath samples will be taken as soon as possible. As noted above, an early awareness of animals being sick (perhaps even before physical signs associated with progression of the disease become apparent) enables treatment to begin more quickly than might otherwise be the case. Further, sick animals may be separated from the herd, which would be expected to prevent transmission of the disease throughout the herd. As would be appreciated, such factors are key for making better business decisions on farm and would be expected to result in improved economic outcomes.

In the present invention, it is important that a sample of ambient air be taken in order for the method to be able to discriminate between VOCs that may be present in the ambient air and VOCs in the bovines' breath samples. In effect, the ambient air provides a “Baseline” for any given location and increases the certainty that any detected biomarkers are indeed present in the bovine's breath.

The process of identifying biomarkers which are represented in a GC-MS dataset by a given retention time (GC) and M/z (mass) is presently undertaken by reference to the National Institute of Standards and Technology (NIST)'s electron ionization library. This database is the product of an ongoing comprehensive evaluation and expansion of the world's most widely used mass spectral reference library. Identification using the NIST library provides a score as to the likelihood of the compound being accurately identified with high confidence.

Biomarkers identified in the breath samples of cows diagnosed with BRD (confirmed by post mortem serology) and characterised using the NIST Mass Spectrometry Data Centre include nitropropane, furan, acetic acid, ethoxy-1-methylethyl ester, propanoic acid ester, ethylamine and cathinone. The inventors have found that that, of these biomarkers, the amounts of 2-nitropropane, furan, acetic acid, ethoxy-1-methylethyl ester and propanoic acid ester increased in the breath samples of sick bovine, and that the amounts of ethylamine and cathinone decreased in the breath samples of sick bovine. Each of these biomarkers will be briefly discussed below, along with reasons why the inventors speculate that they might be relevant to BRD and other respiratory diseases.

### 2-Nitropropane

Nitropropane, also known as 2-nitropropane, is an organic colourless compound with the molecular formula C₃H₇NO₂. Exposure to nitropropane is known to trigger an immune response in the body, with the immune system producing antibodies against nitropropane in response to such an exposure. 2-nitropropane is a pharmacological agent that is used for the treatment of bacterial infections through the inhibitory effect of the enzyme cyclooxygenase-2 and prostaglandins production. 2-nitropropane has been found in alveolar air of patients with pancreatic cancer.

The presence of nitropropane in the breath samples of bovines could potentially be a response of the disease that create various biochemical and physiological effects including inflammatory, antiviral and antibacterial properties Thus, upregulated 2-nitropropane in immunocompromised cows is believed to be capable of detecting newly infected as well as those already sick animals.

### Furan

Furan is a heterocyclic organic compound with a chemical formula of C₄H₄O. Furan has been identified as a significant VOC of primary lung cancer in alveolar breath of patients diagnosed with lung cancer, which might indicate a relationship between furan and respiratory diseases. A specific study aiming at volatile emission from cell cultures with human respiratory disease virus identified three furan derivatives: furan, 2,3-dihydro-furan, and tetrahydrofuran. as biomarkers.

Viruses and bacteria involved in bovine respiratory disease (and other respiratory virus) might potentially enhance furan which can destabilize the animal's immune system and stimulate immune response. Furan is known to be capable of eliciting an immune response in the body and is an essential part of the antigen antibody reaction process.

### Acetic Acid, Ethoxy-1-Methylethyl Ester

Acetic acid, ethoxy-1-methylethyl ester is also known as isopropyl acetate. Isopropyl acetate is an ester, an organic compound which is the product of esterification of acetic acid and isopropanol. Although acetate is known as dietary metabolite, recent research suggests that it may also play a role in pathogenesis, ranging from metabolic syndrome, cancer, and respiratory diseases. Isopropyl acetate has also been found in high amount in people infected with tuberculosis induced respiratory disease and links have been postulated between pulmonary tuberculosis and isopropyl acetate.

As BRD is a complex disease involving both virus and bacteria, an upregulation of isopropyl acetate in the diseased cows is likely produced by the microorganism disrupted cells due to the infection. Both viruses and bacteria produce acetate in modulating immune response, linking diet to the regulation of inflammation through acetyl-CoA signalling pathways. This suggests that enhanced isopropyl acetate in the diseased cow may be emitted by cells that are linked with immune response to pathogen.

### Propanoic Acid Ester

Propanoic acid ester is a colourless volatile liquid with a pineapple-like odour. The presence of propanoic acid ester has been observed in the breath of patients with lung cancer and respiratory disease. Propanoic acid ester is known to be associated with pathophysiological actions, for example in case of bowel syndrome, ulcerative colitis including metabolic disorders. Like acetate, propanoic acid ester creates an inflammatory response and initiates calcium mobilization in peripheral immune cells. It also influences intracellular pH, mitochondrial function and gene expression to promote antimicrobial peptide expression. Esters of propionate have anti-inflammatory effect on immune cell functions. In murine it was found that propionate enhances T-cell dependent cytokine milieu, beneficial effect on respiratory diseases. Thus, increased level of propionate ester in diseased bovines may be an indicator of accelerated immune response due to the disease.

### Ethylamine

Amines are the building blocks of all amino acids, and ethylamine is a two-carbon primary aliphatic amine. Amino acids and amine levels have been shown to be lower in cattle affected with BRD. The activation and stimulation of the immune system during an infection in infectious disorders causes a fall in most plasma amino acid levels. This decrease in amino acids and amines is due to the increased need for production of immune system cells, such as leukocytes, T-cell which can require high levels of specific amino acids. T cells, a type of immune cell that can target and destroy virus-infected cells, may be able to give long-term protection against viral infection, even if antibodies become less effective. When combined with healthy human peripheral blood mononuclear cells, ethylamine has been found to promote a 15-fold increase of T-cells. Histamine, produced from ethylamine, plays a crucial role in the inflammatory response by producing immune cells (basophils and by mast cells).

Thus, the significant decrease in ethylamine observed by the inventors might be explained in two ways: first, BRD-affected animals have lower feed intake in general, and lower dietary protein intake via feedstuffs exacerbates increased nitrogen usage by the body, resulting in amine deficiency, particularly ethylamine, in the bloodstream. Secondly, given the above evidence an acute deficiency in ethylamine in BRD affected cows is observed because such has been used to produce immune cell mediated host defence and immunoregulation.

### Cathinone

Cathinone is an intermediate metabolite in the biosynthesis of cathine and is structurally related to the synthetic drug, amphetamine. Although there is little information on the mode of action of cathinone in animal body, it has been known to be involved in phosphorylation, signal transducers, stress sensors, and body defence mechanism. Increased weight of the two immune organs as an indicator for immune stimulating potential cathinone has been observed in albino mice. Further, dose dependent increment in antibody titer and cellular response (T cell and B cell) has been documented in mice treated with the cathinone compound. The reduced cathinone in BRD affected cattle in the present study might be due to the excessive use of it in the immune defence system of diseased animals; a mechanism similar to ethylamine.

The inventors note that these data are preliminary and that further research (which is currently underway) will be required to confirm whether additional VOCs might also be biomarkers that are significant to the detection of BRD (and other respiratory diseases, such as bovine pestivirus).

The inventors note that these biomarkers (or at least some of these biomarkers) may be metabolites of one or more metabolic processes in the bovine. Thus, other metabolites (i.e. in addition to the specific biomarkers described above) produced in metabolic processes which are affected by a bovine having a respiratory disease may be useful in the context of the present invention. An increase in the amount of the biomarker(s) in the breath sample may be because it is a metabolite of a metabolic process that is upregulated in animals having BVD (for example). Similarly, a decrease in the amount of the biomarker(s) in the breath sample may be because it is a metabolite of a metabolic process that is downregulated in animals having BVD. An increase in the amount of one or more of the biomarkers in combination with a decrease in the amount of other of the biomarkers may, for example, be indicative of the progression of BVD in the animal, or the severity of the disease, both of which are clinically significant and the detection of which thus commercially significant.

The method of the present invention comprises the step of comparing the detected amounts of the one or more biomarkers to a predetermined biomarker profile. The predetermined biomarker profile may be specific to a particular species of bovine, if differences in the biomarkers and the amounts of the biomarkers are found to exist. In effect, the predetermined biomarker profile provides a baseline against which the relative increase or decrease in the amounts of the biomarkers in the animal's breath can be assessed.

The predetermined biomarker profile may be obtained in any suitable manner. Typically, the predetermined biomarker profile would comprise (or be defined by) a data set including an accumulation of detected amounts of the one or more biomarkers in breath samples from the same species of bovine, and which have a known disease state (even if this condition is applied to the data after its sampling and subsequent analysis). For example, tens, hundreds or even thousands of animals' breath samples may be analysed to determine their biomarker content, with the results of those analyses being combined with the animals' disease states (possibly using subsequently obtained data) to provide the predetermined biomarker profile. A specific predetermined biomarker profile for bovine, and the method used to create this profile, will be described in further detail below.

Any suitable technique may be used to detect the amounts of the biomarkers in the animal's breath sample. In the proof of concept trials conducted to date, sample collection and analysis have been separately performed. Given that one of the intended applications of the present invention is for the field testing of livestock, however, techniques that use portable equipment that is robust, simple to operate and reliable, would be preferred. For example, the inventors believe that “Electric nose” sensors of the kind described below might be used in the present invention, even though commercially viable sensors are not yet readily available. Regardless, equipment that utilises such point of use analytical methods are typically secondary in nature, i.e. they need to be calibrated against a known analytical reference method of high precision and accuracy.

The standard method of gas sample analysis is gas chromatography-mass spectrometry (GCMS), used around the world in analytical laboratories for medical, forensic and many other industrial applications. GCMS combines the separating capabilities of gas chromatography with the molecular identification power of mass spectrometry. Whilst this equipment is generally not portable, requires skilled operators, is expensive and requires significant maintenance, it may be used to validate a selection of results in order to establish or maintain a calibration of a simpler detection device, such as an “Electric nose” sensor of the kind described below. Furthermore, some emerging technologies utilise GC-MS and may provide a new generation of analytical tools that are far smaller than conventional GC-MS instruments. Such technology, if developed, may allow for GC-MS techniques to be practical for use “in-field”.

GCMS outputs two separate but highly linked data outputs; a chromatogram, which is a multivariate fingerprint of the samples as measured by the GC system as total intensity vs. time. A single chromatogram is generated per sample measured and the patterns in the chromatogram were anticipated to be indicative of disease when assessed using multivariate pattern recognition algorithms. A mass spectrum may be generated for every point measured in the chromatogram and the length of the mass spectrum determined by the highest molecular weight compound detected in the sample. In general, the mass spectrum is usually interpreted when a peak in the chromatogram is determined to be important and its chemical identification is to be established.

In some embodiments therefore, detecting an amount of the one or more biomarkers may comprise chromatographically resolving the breath sample (e.g. by gas chromatography or analytical techniques involving GC such as GC-MS). Whilst GC equipment would generally not be portable and might not be appropriate for all applications of the present invention, it would be very useful in establishing and maintaining the predetermined biomarker profile, as well as for calibrating and maintaining more portable electronic devices (e.g. sensors such as the “Electric nose” sensors described below). The inventor also notes that there may be occasions when the accuracy and reliability of GC systems make them commercially viable (e.g. for larger farms).

Once collected, the detected amount(s) of the biomarker(s) in the breath sample would usually need to be analysed before any determination of the animal's disease state can be provided. Any suitable data analysis methodology (e.g. of the mass spectral dataset) that is compatible with the detection techniques described herein may be used in the present invention.

For example, as noted above the generated mass spectra dataset produced for a plurality of points of a sample that has been resolved by gas chromatography is complex and would usually require the use of multivariate analysis (MVA) techniques. MVA techniques are well suited to the analysis of highly multidimensional data and have previously been used in the agriculture, pharmaceutical and petrochemical industries for real time predictions and early event detection, as well as in major processing industries for the extraction and interpretation of complex patterns in data that cannot be analysed by simple statistical routines.

Three particular methods of analysis are expected to be useful for the evaluation of data obtained in accordance with the present invention, namely principal component analysis (PCA), Partial Lest Squared Discriminant Analysis (PLS-DA) and Partial Least Squares Regression (PLSR). Examples of these methods of analysis being used in the context of the present invention will be described below. These are standard and well documented methods, known as multivariate methods as they assess more than one variable at a time.

PCA is a method of analysis which provides a highly visual environment for detecting patterns in complex data, such as the total ion chromatographs (TICs) generated by GC-MS. It allows an analyst to see if there are any within group variations (e.g. bovines with/without BVD) and any time dependent changes in the groups. The main advantage of PCA is that it is highly interpretable and can be validated.

PLS DA is an alternative method to PCA that provides more direct modelling capabilities when the classes of data (e.g. data from a particular sampling time) are known. Time of sampling (e.g. day) can then be used as a class to discriminate between any changes on a day.

PLSR is a multivariate regression method that allows for the development of a predictive model utilising multiple inputs from a sensor. In the context of the present invention, the changes in biomarkers in the GC-MS data may be calibrated against the responses generated by sensors such as the “Electronic nose” sensor described below. Like PCA, PLSR is also highly visual, is interpretable and can be validated, which can provide much more reliability and integrity to the prediction results generated by multivariate models compared to other methods of analysis. PLSR also has inbuilt diagnostics to ensure that prediction results are valid.

The applicant's earlier international (PCT) patent application no. PCT/AU2021/050481 (WO 2021/232110), the contents of which are hereby incorporated by reference, describes these analysis techniques in the context of determining whether an animal is pregnant.

In other embodiments, an amount of the one or more biomarkers may be detected using a sensor, preferably a portable sensor and even more preferably a hand-held sensor coupled to a sampling device such as that disclosed in PCT/AU2020/050318. In such embodiments, detecting an amount of the one or more biomarkers may comprise contacting the breath sample with a plurality of sensors that are configured to sense the presence of the biomarker(s), whereby a (predetermined) cumulative response of the sensors is indicative of the bovine having bovine respiratory disease. Such sensors will be described in further detail below.

The present invention thus also provides a sensor for determining whether a bovine has a respiratory disease, such as bovine respiratory disease. The sensor comprises a detector for detecting an amount of one or more biomarkers in a breath sample from the bovine, and an analyser for analysing the detected one or more biomarkers and comparing with a predetermined biomarker profile, whereby a correlation between the detected one or more biomarkers and the predetermined biomarker profile is indicative of the bovine having the respiratory disease.

In some embodiments, the detector may comprise a plurality or array of sensors that are configured to detect the one or more biomarkers, whereby a (predetermined) cumulative response of the plurality of sensors is indicative of the bovine having bovine respiratory disease.

For example, the “Electronic Nose” sensor, sold under the brand Cyranose® by Sensigent, Los Angeles, USA, is a handheld chemical vapour sensing instrument designed to detect and identify complex chemical mixtures that constitute aromas, odours, fragrances, etc. The Cyranose® sensors have been used in industries including petrochemical, chemical, food and beverage, packaging materials, plastics, pet food, pulp and paper and medical research. The Cyranose® sensors utilise an array of detectors that are sensitive to chemical species incident upon them as well as advanced pattern recognition algorithms to detect and recognize the chemical vapour of interest via its “Smellprint”. In combination, these technologies enable rapid detection and identification of substances based on their chemical profile, as visualized by the smellprint.

The inventor expects that a sensor such as a Cyranose® sensor will be capable of being adapted to detect the biomarkers described herein in a breath sample from the animal and hence to determine whether the animal has a respiratory disease such as BRD. So-called “Electronic Nose” sensors should therefore be able to be configured to be suitable for use in the field and for detecting any particular combinations of biomarkers which are determined in accordance with the present invention to be indicative of the animal having BRD.

Thus, a system capable of detecting a respiratory disease such as BRD might be provided through the use of a breathalyser-type device that captures a sample of breath from the animal using a sampling device specifically made for a non-expert user to capture a breath sample without causing distress to the animal. The breath sample is then analysed using a sensor capable of determining individual components or volatiles present in the breath sample and, should the correct mix of components that signify the animal having BRD, a positive result is provided. The commercial relevance of such a simple to perform test, which provides an immediate and on-farm diagnosis, is immediately apparent.

The inventors believe that their ongoing research, utilising proven laboratory based research grade analysis technology, will enable them to better understand the patterns in biomarkers over time. Their further work aims to correlate the results obtained with a portable, on-farm device that will provide timely measurements in the field. Based on the data obtained thus far, some of which is described below, and on previous research and experience, the inventors expect that on-farm testing for a respiratory disease such as BRD using the method of the present invention is plausible.

The inventors also expect that the results of the testing regimen described herein can be utilised in other applications. For example, the sensor described herein may automatically communicate the results to farm management software. Such results could also be integrated into automated animal handling equipment, for example in drafting sick from healthy animals.

### Examples

The inventors have conducted “Proof of concept” studies, where breath samples from healthy and sick cattle (i.e. confirmed suing conventional techniques) were analysed to confirm that biomarkers related to BRD could be detected. The results of these studies are described below.

### Materials and Methodology

### Animals, Ethics & Location

The study was carried out at Kilara Feedlot, in Quirindi NSW, in collaboration with on-site veterinarians trained in using the Agscent™ breath sampler (https://agscent.com/). A total of 12 bovines were used in this study. The experiment was conducted in accordance with the Australian code for the care and use of animals for scientific purposes and all procedures were approved by the Secretary's Animal Care & Ethics Committee (TRIM19/2523).

### Materials

The following materials and equipment used in this study were obtained from commercial sources. The 2-litre PTFE gas-sampling bags connected with one-way inflow valves were purchased from Scentroid (Canada). Soft silicone funnel-like breath snouts designed and owned by Agscent were used to capture exhaled breath. Breath samples were extracted into sorbent tubes which were obtained from SulfiCarb, Markes International Limited, UK. A hand-held pump used to extract gas was obtained from SKC, USA.

### Training

An initial trip to Quirindi was conducted in order to scope the feedlot research site and conduct training on record keeping, breath sampling, maintaining instruments and made sure all participants were comfortable with using the Agscent™ breath sampler and the experimental protocol. On-site veterinarians were instructed on the storage/handling/shipping of samples to Agscent for further breath volatile analysis via gas chromatography-mass spectrometry (GC-MS).

### Experiment Design

Mixed breed steers (Bos taurus) were sourced to the feedlot through normal procurement practices at high risk times of year (Autumn) over two successive years. The sample population consisted of 6 steers infected with BRD and 6 control animals.

A clinical information system (CIS) was used, which ranges from 1 (healthy) to 4 (extremely ill) with the specific criteria used for each level. The following criteria was used to determine each CIS level: CIS 1=normal behaviour and appetite, CIS 2=slight illness, mild depression, and/or a cough, CIS 3=moderate illness, severe depression, laboured breathing, and/or cough, and CIS 4=severe illness, where animals may be moribund or have little response to human approach. Clinical illness scores were conducted every 12 hours by a trained feedlot pen checker.

Case definition based on “Visual Symptoms+Fever+Lung Pathology” is set out in Table 1.

TABLE 1
Case Definitions

| | |
|---|---|
| Control (i.e. Healthy | CIS never >1 at any time point; Temp less than |
| bovine) | 40° C. at last evaluation, lung lesions less |
| | than 5% at autopsy |
| Case (i.e. infected | at least one CIS equal or greater than 3; Temp |
| bovine) | greater or equal to 40° C. at last evaluation, |
| | lung lesions greater or equal to 5% at autopsy |

### Experimental Procedure

The Agscent™ breath sampler was used with custom made PTFE collection bags designed with a one-way valve and an exit mechanism which could easily and securely be attached to a tube which allowed for safe and efficient decanting of the sample onto the sorbent tube for subsequent analysis by trained specialists at the Commonwealth Scientific and Industrial Research Organisation (CSIRO) in Canberra, Australia.

The sorbent tube used 1 litre of breath, leaving the same sample from the same cow available also for the sensor device described in the following Example (thus eliminating the need for duplicate samples). Sample bags were transported to CSIRO in Canberra for GC-MS analysis.

21 samples in total were analysed; 8 of which were ambient air, 7 of which were control (i.e. healthy) animals and 6 of which were from steers positively diagnosed with BRD (all animals were euthanised to confirm the presence and absence of the disease via pathology testing).

### GC-MS Running Method

A gas chromatograph (Bruker 451 Model GC, Bruker Daltonik Inc., USA) using a GC capillary column ZB-5MS (Phenomenex Australia Pty Ltd.) 30 m in length, 0.25 mm ID, and 0.25 μm film thickness was used with the following temperature program: initial temperature 35° C. and held for 5 min, ramped to 180° C. at 5° C. min⁻¹ then ramped to 250° C. at 8° C. min⁻¹. The final temperature of 250° C. was held for 10 min. The total run time for the analysis was 53 min. Helium carrier gas flowed at a rate of 0.8 mL·min⁻¹.

A single quadrupole mass detector (Scion SQ, Bruker Daltonik Inc., USA) set with a full scan detection covering the ion mass range from 35 to 350 m/z, with positive polarity. Data was generated as raw .XMS files from Bruker. The files were converted into .CDF format using OpenChrom Edition software for statistical analysis

### Statistical Methods

Data was analysed in a manner similar to that described in PCT/AU2021/050481, the contents of which are hereby incorporated by reference. Principal component analysis (PCA), PLS DA and Partial Least Squares Regression (PLSR) were used to identify volatile biomarkers and their relationship to BRD cases and controls. The samples were only categorised as case vs control vs ambient air.

Principal Component Analysis (PCA) is a method of analysis which provides a highly visual environment for detecting patterns in complex data, such as is the case for the total ion chromatographs (TICs) generated by GC-MS. It allows an analyst to see if there are any within group variations, for instance pregnant vs. non-pregnant animals in the applicant's earlier research, as well as a diagnosis of (and potentially the stage of) BRD in this current research, as well as any time dependent changes in the groups. The main advantage of the method of PCA is that it is highly interpretable and can be validated. Partial least squares DA is an alternative method to PCA that provides more direct modelling capabilities when the classes of data to discriminate between any changes between samples.

### Example 1—Proof of Concept

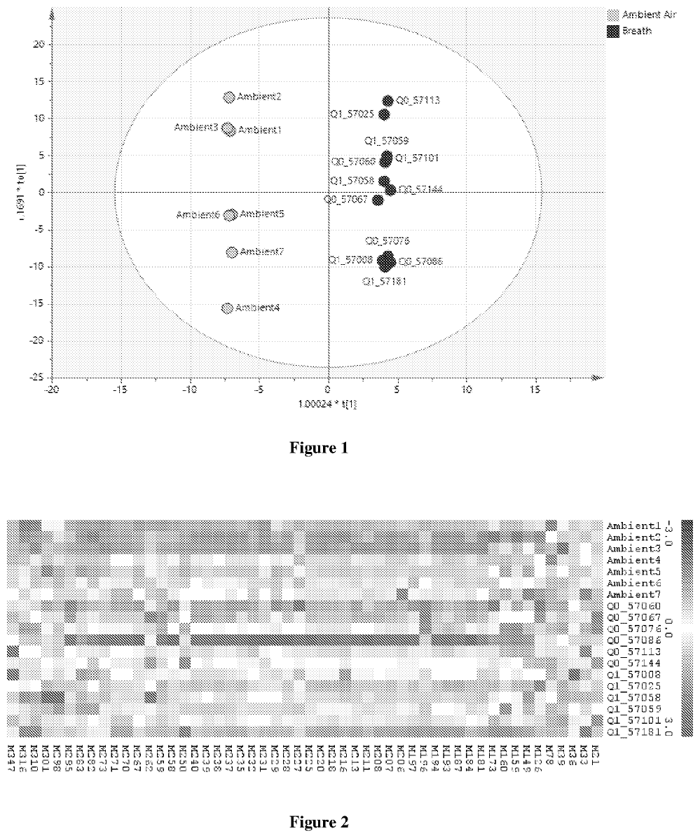

Breath samples, obtained as described above, were analysed by GC-MS. The data obtained by the inventors confirms that breath samples (Case and Control) are significantly distinct from ambient air, following analysis of the GC-MS dataset. This analysis is represented in FIG. 1, which is a score plot of ambient air and breath sample obtained using an OPLS model. OPLS explained most of the variance in the data (R2: 0.999; Q2:0.457), which shows that breath sample could be discriminated easily (qualitatively Y variable). FIG. 1 shows that there is significant separation between the bovines' breath and ambient air.

It is worth noting that many molecules that are found in breath are also found in the environment, in ambient air. The analysis here is to demonstrate that the biomarkers being attributed to BRD are distinctly found in animals and not in the ambient air. This will be important in further development of the technology, as the ambient air will be used to calibrate any sensor technology to ensure that only metabolites relevant to the animal are included in the analysis.

Using a heat map visualisation, FIG. 2 shows significant VOCs found in the bovine breath samples (threshold P-value of 0.05 with Bonferroni correction). A blue cell indicates a decrease in the relevant VOC and a red cell indicates an increase. Sample size: 7—ambient air and 12—breath sample (6—control and 6—case).

Step two in the analysis was to model potential significant differences between Case (i.e. breath samples from infected bovine) and Control (i.e. breath samples from healthy bovine) biomarkers using GC-MS. Whilst the small numbers of animals and correspondingly small dataset can only be interpreted as ‘indicative’ of potential significance, and further research is required to confirm these initial findings, the results are very positive and certainly do provide a proof of concept.

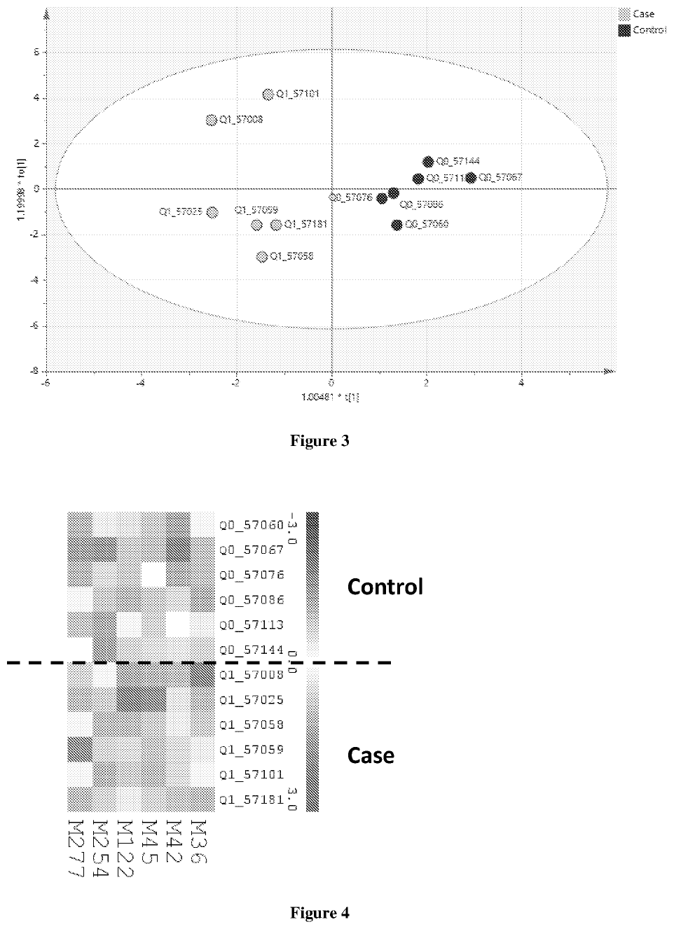

FIG. 3 represents the score plot of Control and Case (by OPLS model). OPLS explained most of the variance in the data (R2: 0.9; Q2:0.693) which show that disease sample could be discriminated easily (qualitatively Y variable). In each case, n=6. The R2 value in this analysis shows how strong the model is in its ability to discriminate between diseased (i.e. Case) and non-diseased (i.e. Control) animals. An R2 value of 0.9 shows a very high level of statistical discrimination using a regression model, showing that the identified VOCs are those which contribute most strongly to the model. The Q2 scores show these specific VOCs are highly correlated to the state of the animal.

When considering the differences between the VOCs which are statistically significantly different between Case and Control, the inventors observed that some increase in representation and others decrease. FIG. 4 shows a heatmap to demonstrate the key biomarker metabolites of significance related to disease, threshold P-value of 0.05 with Bonferroni correction. Blue cell: decrease; Red cell: increase. In each case, n=6. The inventors found that M36, M45, M122 and M254 are up-regulated in diseased samples, while M42 and M 277 are down-regulated in diseased samples.

### Example 2—Identifying Volatile Organic Compounds from GC-MS

The process of identifying VOCs in the bovines' breath samples which are represented by retention times (GC) and M/z (mass) at this early stage was done with reference to the National Institute of Standards and Technology (NIST)'s electron ionization library. This database is the product of an ongoing comprehensive evaluation and expansion of the world's most widely used mass spectral reference library.

Identification using the NIST library provides a score as to the likelihood of the categorisation of the VOC being accurately identified, with a score of over 700 demonstrating high confidence. Further confirmation of identification is generally conducted using chemical standards, however at this early stage (pilot project with few cases), reference to the NIST library is appropriate and is the basis of our findings.

The full table of potential VOCs was considered, with a reduced list of the most likely compound identifications and their biological rationale based on existing literature is as follows. These equate to both up and down regulated metabolites 36, 45, 122 & 254 and 42 & 277 (see FIG. 4):
2-nitropropane
Furan
Acetic acid, ethoxy-1-methylethyl ester
Propanoic acid ester
Ethylamine
Cathinone

### Example 3—Preliminary Trials Using a Nanofiber Sensor

A herd of yearling heifers at Bungendore NSW identified as unwell and possibly displaying symptoms of BRD or BVD (bovine viral diarrhea) were tested. The Agscent™ breath collection device was used to collect breath from approximately 10 animals which appeared to be healthy and approximately 20 animals which appeared to be in a poorly/sick condition and displaying symptoms of BRD or BVD.

Assessment of each animal was against the following diagnostic chart:

| Normal | BVD | BRD |
|---|---|---|
| Normal breath | Diarrhoea | Depressed |
| (10-30/min) | | |
| Alert and aware of | Bruce | High rate of breath |
| surrounding | | |
| Clear eye, no discharge | Bleeding from wound | Nasal discharge |
| Erect and moving ears | Red eye | Eye sleepy/closed |
| Frequently lick | Dirty tail or flank | Cleaning nostril |
| nose & coat | | |
| Chewing cud | Skin disease | Head down |
| Eat normally | Ocular discharge | Away from the mob |
| | Depressed appearance | Less feed intake |
| | Soft cough | Droll from the mouth |
| | Lameness | Shallow breathing |
| | Loss of appetite | Coughing |
| | Drooling from mouth | |

### Animals, Ethics & Location

A total of 30 non-pregnant Angus heifers were used in this study. The experiment was conducted in accordance with the Australian code for the care and use of animals for scientific purposes and all procedures were approved by the Secretary's Animal Care & Ethics Committee (TRIM19/2523). Animals used in this study included animals which were clinically normal and those who were clinically unwell. Their comfort and tolerance of the procedure was monitored throughout the study.

### Materials

The following materials and equipment used in this study were obtained from commercial sources. The 2-litre PTFE gas-sampling bags connected with one-way inflow valves were purchased from Scentroid (Canada). The nanofiber sensor was also obtained from Scentroid (Canada). Soft silicone funnel-like breath snouts designed and owned by Agscent were used to capture exhaled breath. The laptop, power inverter, elastic bands, esky, first aid kit, tool kit and other stationaries that were used in this study were obtained locally. A harness was also utilised to hold the head of animals still as required.

### Method

A total of 30 heifers were brought into the yards for breath collection. Heifers were placed in the head hold and were restrained using a halter if required. A 2-litre sample of breath was collected from 12 cows that presented as healthy cows and which had not previously been identified as sick, using a 2-litre PTFE bag connected to a one-way valve, CO₂ diverter capturing breath at CO₂ levels (opening at 1%, closing at 2%) with a breath snout. A 2-litre sample of breath was also collected from 18 cows that previously had presented as unwell, possibly suffering from BRD or BVD and had been treated. The bags were tightly screwed to avoid escape of the breath.

2-litre samples of breath in PTFE bags were analyzed using the Nanofiber Sensing system Version 1.0 (SCENTROID Future of Sensory Technology, IDES CANADA INC.) immediately following collection. The Nanofiber Sensing system is a combination of 16 different Nanofiber chemical sensors that respond to gaseous compounds and allows for real time monitoring of complex gaseous mixtures. In between each breath sample, the Nanofiber sensor sampled filtered air between each breath sample. The Nanofiber sensor data was electronically saved as an excel file for further analysis.

A total of 61 samples (breath: 9 healthy, 18 sick and treated, and another 3 healthy for calibration; filtered air: 28 samples, 2 for calibration, and 1 for warm-up) were taken in this trial and were analysed by the Scentroid Nanofiber sensor for a total duration of 156 minutes. An average of the sampling period between 20-30 seconds for each channel was used for analysis in this trial.

### Statistical Analysis and Data Presentation

Supervised multivariate analysis of sensor data was carried out by orthogonal projection on latent structure (OPLS), using the breath of healthy vs sick & treated cows as predicted qualitative Y variables and VOCs as predicting X variables. The absence of statistical outliers was first checked using a principal component analysis (PCA) to verify that no data point was outside the 99% confidence Hostelling region. The goodness of the OPLS model was appreciated using the determination coefficient R² and the predictive power was quantified by the cross-validated determination coefficient, Q². The significance of the statistical OPLS model was tested using a c² comparison with a random model (average±random error), and the associated P-value (PCV-ANOVA) is reported. Univariate analysis of statistical classes was performed using a one-way ANOVA (Bonferroni test), with a threshold of P=0.05.

### CONCLUSIONS

The main aim of this trial was to differentiate between the two groups of cows' breath: ‘healthy’ and ‘was sick & treated’. During this study there was a failure of the breath capturing device in the second half of the study. This resulted in the ingress of ambient air, resulting in analysis between the healthy and sick & treated groups insignificant in those cows where this occurred. Where ambient air did not ingress into the sample, healthy vs was sick & treated groups from the early measured samples could clearly be separated into two groups: healthy group vs sick and treated group using OPLS.

These results, although preliminary in nature and incomplete, demonstrate that a commercially available nanofiber sensor is able to discriminate between animals that were sick and animals that were well. The inventors thus expect that using an “e-nose” sensing device is likely to be effective in detecting the known VOC's which we have analysed during the previous GC-MS trials, and hence provide a diagnosis of BRD.

Indeed, the inventors expect that it will be possible to use the Agscent™ breath sensing device (in conjunction with new sensors that they are currently trialing) to potentially screen all animals prior to admission into a feedlot, with those indicating biomarkers of respiratory diseases such as BRD being either excluded or segregated and monitored. This would reduce the contagious effect of the disease upon induction and further reduce the prevalence of BRD (etc.) in feedlots in Australia.

As described herein, the present invention provides methods for determining whether a bovine has bovine respiratory disease. Embodiments of the present invention would provide a number of advantages over existing disease detection methods, including:
short- and long-term business benefits, including:
   cost reduction in testing per animal;
   introduction of precision farming aspects through better feeding and growth programs;
screening prior to moving animals from one farm to another would prevent infections spreading;
early detection allows for animals to be separated to see if they develop the disease further and to be treated more effectively according to their own development path of the disease;
repeated use of the scanning device could track the development of the disease or demonstrate when animals have recovered;
the simplicity of a hand held system would reduce labour costs and make disease detection a non-invasive process requiring much less technical expertise;
automation of breath collecting could be paired with sensor incorporation and remove the human element of both breath collection and diagnosis; and
customised breath sampling device (and associated algorithms) could function as a non-invasive, simple and easy to use BRD screening device in feedlots, thereby improving animal welfare and farm productivity.

It will be understood to persons skilled in the art of the invention that many modifications may be made without departing from the spirit and scope of the invention. All such modifications are intended to fall within the scope of the following claims.

It is to be understood that any prior art publication referred to herein does not constitute an admission that the publication forms part of the common general knowledge in the art.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1. A method for determining whether a bovine has a respiratory disease, the method comprising:
detecting an amount of one or more biomarkers in a breath sample from the bovine, the detected amount of the or each biomarker defining a detected biomarker profile; and
comparing the detected biomarker profile with ambient air and to a predetermined biomarker profile, whereby a correlation between the detected and predetermined biomarker profiles is indicative of the bovine having the respiratory disease.

2. The method of claim 1, wherein the predetermined biomarker profile comprises an accumulation of detected amounts of the one or more biomarkers in breath samples from bovines diagnosed as having the respiratory disease.

3. The method of claim 1, wherein detecting the amount of the one or more biomarkers comprises chromatographically resolving the breath sample.

4. The method of claim 3, wherein detecting the amount of the one or more biomarkers further comprises producing mass spectra for a plurality of points of the chromatographically resolved breath sample, whereby a mass spectral dataset which defines the detected biomarker profile is produced.

5. The method of claim 4, wherein comparing the detected biomarker profile with ambient air and to a predetermined biomarker profile comprises analysing the mass spectral dataset.

6. The method of claim 5, wherein the mass spectral dataset is analysed using a multivariate analysis.

7. The method of claim 5, wherein the analysis is a partial least squared discriminant analysis.

8. The method of claim 1, wherein detecting the amount of the one or more biomarkers comprises contacting the breath sample with a plurality of sensors that are configured to sense the one or more biomarkers, whereby a cumulative response of the plurality of sensors is indicative of the bovine having the respiratory disease.

9. The method of claim 1, wherein the one or more biomarkers comprise biomarkers selected from one or more of the following: 2-nitropropane, furan, acetic acid, ethoxy-1-methylethyl ester, propanoic acid ester, ethylamine and cathinone.

10. The method of claim 9, wherein the amount of one or more of 2-nitropropane, furan, acetic acid, ethoxy-1-methylethyl ester and propanoic acid ester is elevated in bovine having the respiratory disease.

11. The method of claim 9, wherein the amount of one or both of ethylamine and cathinone is reduced in bovine having the respiratory disease.

12. The method of claim 1, wherein the breath sample is collected from the bovine's nose.

13. The method of claim 1, wherein the respiratory disease is selected from the group consisting of bovine respiratory disease, bovine viral diarrhea and bovine pestivirus.

14. A method for screening a population of bovines for the presence of a respiratory disease, the method comprising:
detecting an amount of one or more biomarkers in an air sample taken in close proximity to the population of bovines, the detected amount of the or each biomarker defining a detected biomarker profile; and
comparing the detected biomarker profile with ambient air and to a predetermined biomarker profile, whereby a correlation between the detected and predetermined biomarker profiles is indicative of one or more bovines in the population having the respiratory disease.

15. A sensor for determining whether a bovine has a respiratory disease, the sensor comprising:
a detector for detecting an amount of one or more biomarkers in a breath sample from the bovine, and
an analyser for analysing the detected one or more biomarkers and comparing with a predetermined biomarker profile, whereby a correlation between the detected one or more biomarkers and the predetermined biomarker profile is indicative of the bovine having the respiratory disease.

16. The sensor of claim 15, wherein the detector comprises a plurality of sensors that are configured to sense the one or more biomarkers, whereby a cumulative response of the plurality of sensors is indicative of the bovine having the respiratory disease.

17. The sensor of claim 15 when used in a method for determining whether a bovine has a respiratory disease, the method comprising:
detecting an amount of one or more biomarkers in a breath sample from the bovine, the detected amount of the or each biomarker defining a detected biomarker profile; and
comparing the detected biomarker profile with ambient air and to a predetermined biomarker profile, whereby a correlation between the detected and predetermined biomarker profiles is indicative of the bovine having the respiratory disease.
